(19) United States
(12) Patent Application Publication
(10) Pub. No.: US 2026/0191510 A1
(43) Pub. Date: Jul. 9, 2026

(54) AUTOMATIC MEASUREMENT POINT DETECTION FOR ANATOMY MEASUREMENT IN ANATOMICAL IMAGES

(71) Applicant: KONINKLIJKE PHILIPS N.V., EINDHOVEN (NL)

(72) Inventor: Earl Monroe Canfield, II, New Braunfels, TX (US)

(21) Appl. No.: 19/129,811

(22) PCT Filed: Nov. 8, 2023
(86) PCT No.: PCT/EP2023/081129

§ 371 Date: May 14, 2025

Related U.S. Application Data
(60) Provisional application No. 63/425,539, filed on Nov. 15, 2022.

(51) Int. Cl. A61B 8/00; G06T 7/60; G06V 10/774; G06V 10/776; G06V 10/82

(52) U.S. Cl. CPC A61B 8/5223; A61B 8/463; G06T 7/60; G06V 10/82; G06T 2207/10016; G06T 2207/10132; G06T 2207/20081; G06T 2207/20084; G06V 10/774; G06V 10/776; G06V 2201/03

(57) ABSTRACT

A system includes a display and a processor configured for communication with the display and a medical imaging device. The processor is configured to receive an anatomical image frame obtained by the imaging device during live imaging, and identify a plurality of measurement points of an anatomical feature in the anatomical image frame. The identification of the first measurement points is performed by a neural network trained to identify where a measurement point is located within an anatomical image frame, such that the measurement points are identified automatically, without a user input. The processor is also configured to generate, based on measurement points, a measurement value of the anatomical feature, and output a screen display based on the measurement value.

### FIELD

The subject matter described herein relates to devices, systems, and methods for automatically locating and measuring features (e.g., anatomical features or pathologies) in during live imaging by a medical imaging device (e.g., an ultrasound probe). For example, a neural network is trained to identify measurement points in anatomical images, which can be used to generate measurements of the features.

### BACKGROUND

Ultrasound imaging is often used for diagnostic purposes in an office or hospital setting. For example, ultrasound is an imaging technique deployed at the point-of-care to aid in evaluation of fetal development. Important anatomical features-such as head circumference, abdomen circumference, and femur length can be obtained in near-real time by a clinician, by freezing the real-time ultrasound video stream at a particular image, placing measurement points on the image, and then either performing manual calculations or relying on software algorithms to perform the calculations based on, for example, the distances between certain measurement points. Measurement points could be two endpoints (e.g., for a measurement value based on the distance of the line between the two points.) The measurement points could also be more than two points. For example, more than two measurement points could define a curve, a curvilinear shape, a closed shape, etc. (e.g., an area, a circumference, a perimeter, etc.). In some cases, measurement points may be referred to as calipers (e.g., in reference to physical calipers that may be used to acquire similar measurements from an infant subsequent to birth). Such measurement processes can be imprecise, labor-intensive, and heavily reliant on the training level of the practitioner.

For example, an ultrasound exam protocol may require the clinician to scan the patient looking for specific anatomy of interest, pause the system, press the appropriate measurement button, and position the measurement calipers to measure the anatomy. The clinician typically will measure 3 or more measurements for each anatomy and average the results to derive an accurate measurement, which accounts for a large portion of the total exam time.

The information included in this Background section of the specification, including any references cited herein and any description or discussion thereof, is included for technical reference purposes only and is not to be regarded as subject matter by which the scope of the disclosure is to be bound.

### SUMMARY

Disclosed is an automatic measurement point detection system using an artificial intelligence/machine learning algorithm (e.g., a neural network). The neural network is trained on anatomical image frames that are human-annotated with measurement caliper positions, and can be used to automatically identify and measure anatomy (depicted in the medical image, such as an ultrasound image or an x-ray image) in real time. For example, in ultrasound imaging, the clinician or other user simply moves the transducer over the anatomy of interest and the trained neural network identifies the anatomy and positions the measurement calipers in real time to automatically calculate the measurement, with no intervention from the user. Instead of the traditional 3 measurements performed during an exam for each anatomy, the automatic measurement point detection system completes hundreds of measurements at real time frame rates. Once measurement convergence is identified, the user interface indicates the most accurate measurement has been achieved.

This automatic measurement point detection system disclosed herein has particular, but not exclusive, utility for measuring the sizes of anatomical features or pathologies in a real-time ultrasound video stream (e.g., live imaging as a clinician moves the ultrasound probe over the body of a patient), as may occur for example in a prenatal ultrasound exam. The automatic measurement point detection system detects features in the individual frames of the video, automatically places measurement points on the video frame, performs calculations based on the measurement points, and generates and displays anatomical measurements based on the calculations.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a system which includes a display and a processor configured for communication with the display and a medical imaging device. The processor is configured to: receive a first anatomical image frame obtained by the medical imaging device during live imaging; identify, while the live imaging is ongoing, a plurality of first measurement points of an anatomical feature in the first anatomical image frame, where the identification of the first measurement points is performed by a first neural network trained to identify where a measurement point is located within an anatomical image frame such that the identification of the first measurement points is performed automatically without a user input to locate the plurality of first measurement points in the first anatomical image frame; generate, based on the plurality of first measurement points, a first measurement value of the anatomical feature for the first anatomical image frame; and output, to the display, a screen display based on the first measurement value. Other aspects include corresponding computer systems, apparatus, and computer programs recorded on one or more computer storage devices, each configured to perform the actions of the methods.

Implementations may include one or more of the following features. In some aspects, the processor is configured to: identify, while the live imaging is ongoing, a plurality of second measurement points of the anatomical feature in a second anatomical image frame obtained by the medical imaging device during the live imaging; and generate, based on the plurality of second measurement points, a second measurement value of the anatomical feature for the second anatomical image frame, where the screen display is based on the first measurement and the second measurement. In some aspects, the second frame is obtained immediately after the first frame. In some aspects, the processor is configured to determine, based on the first measurement value and the second measurement value, whether a convergence of measurement values has occurred, where the processor is configured to provide the screen display based on the determination of whether the convergence of measurement values has occurred. In some aspects, the screen display may include a progress of the convergence of measurement values. In some aspects, to determine whether the convergence of measurement values has occurred, the processor is configured to determine whether the second measurement is smaller than the first measurement. In some aspects, if the convergence of measurement values has occurred, then the processor is configured to select, as a converged measurement value, the larger of the first measurement value or the second measurement value; and where the screen display may include the converged measurement value. In some aspects, the processor is configured to determine whether the convergence of measurement values has occurred based on a difference between the first measurement and the second measurement. In some aspects, the screen display may include: the first anatomical image frame; and the plurality of first measurement points overlaid on the first anatomical image frame. In some aspects, the screen display may include: the first anatomical image frame; and an indication of the anatomical feature overlaid on the first anatomical image frame. In some aspects, the processor is configured to identify, in the first anatomical image frame, an anatomy including the anatomical feature, where the processor is configured to generate the first measurement value based on the identification of the anatomy. In some aspects, the screen display may include the first anatomical image frame and an indication of the anatomy overlaid on the first anatomical image frame. In some aspects, the identification of the anatomy is performed using a second neural network. In some aspects, the first neural network and the second neural network are the same neural network. In some aspects, the system may include the medical imaging device. Implementations of the described techniques may include hardware, a method or process, or computer software on a computer-accessible medium.

One general aspect includes a method which includes receiving, with a processor in communication with a medical imaging device, an anatomical image frame obtained by the medical imaging device during live imaging. The method also includes identifying, while the live imaging is ongoing, a plurality of measurement points of an anatomical feature in the anatomical image frame, where the identification of the measurement points is performed by a neural network trained to identify where a measurement point is located within the anatomical image frame, such that the identification of the first measurement points is performed automatically, without a user input to locate the plurality of first measurement points in the anatomical image frame. The method also includes generating, based on the plurality of measurement points, a measurement value of the anatomical feature for the anatomical image frame, and outputting, to a display in communication with the processor, a screen display based on the measurement value. Other aspects include corresponding computer systems, apparatus, and computer programs recorded on one or more computer storage devices, each configured to perform the actions of the methods.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the automatic measurement point detection system, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a schematic, diagrammatic representation of an ultrasound imaging system, according to aspects of the present disclosure.

FIG. 2 is a schematic diagram of a processor circuit, according to aspects of the present disclosure.

FIG. 3 is a schematic, diagrammatic representation of a radiology video, video stream, or video clip, according to aspects of the present disclosure.

FIG. 4 is a schematic, diagrammatic representation, in flow diagram form, of an example ultrasound video feature measurement method, according to aspects of the present disclosure.

FIG. 5 is a schematic, diagrammatic representation, in block diagram form, of at least a portion of an automatic measurement point detection system, according to aspects of the present disclosure.

FIG. 6 is a schematic, diagrammatic representation, in block diagram form, of at least a portion of an automatic measurement point detection system, according to aspects of the present disclosure.

FIG. 7A is a schematic, diagrammatic overview, in block diagram form, of a training mode for the object detector/analyzer, according to aspects of the present disclosure.

FIG. 7B is a schematic, diagrammatic overview, in block diagram form, of a validation mode for the object detector/analyzer, according to aspects of the present disclosure.

FIG. 7C is a schematic, diagrammatic overview, in block diagram form, of an inference mode or clinical usage mode for the object detector/analyzer, according to aspects of the present disclosure.

FIG. 8 is an example screen display of the automatic measurement point detection system, according to aspects of the present disclosure.

FIG. 9 is an example training image for the automatic measurement point detection system, according to aspects of the present disclosure.

FIG. 10 is an example object detection image of the automatic measurement point detection system, according to aspects of the present disclosure.

FIG. 11 is an example “detected objects” display of the automatic measurement point detection system, according to aspects of the present disclosure.

FIG. 12 is an example inference image generated by the automatic measurement point detection system, according to aspects of the present disclosure.

FIG. 13 is an example object detection image of the automatic measurement point detection system, according to aspects of the present disclosure.

FIG. 14 is an example object detection image of the automatic measurement point detection system, according to aspects of the present disclosure.

FIG. 15 is an example filter control set of the automatic measurement point detection system, according to aspects of the present disclosure.

FIG. 16 is an example training image of the automatic measurement point detection system, according to aspects of the present disclosure.

FIG. 17 is an example object detection image of the automatic measurement point detection system, according to aspects of the present disclosure.

FIG. 18 is an example inference image generated by the automatic measurement point detection system, according to aspects of the present disclosure.

FIG. 19 is an example training image for the automatic measurement point detection system, according to aspects of the present disclosure.

FIG. 20 is an example inference image generated by the automatic measurement point detection system, according to aspects of the present disclosure.

FIG. 21 is an example convergence progress display of the automatic measurement point detection system, according to aspects of the present disclosure.

FIG. 22 is an example convergence progress display of the automatic measurement point detection system, according to aspects of the present disclosure.

FIG. 23 is an example convergence progress display of the automatic measurement point detection system, according to aspects of the present disclosure.

### DETAILED DESCRIPTION

In accordance with at least one aspect of the present disclosure, an automatic measurement point detection system is provided which can measure the dimensions of anatomical features or pathologies in individual frames of a real-time ultrasound video stream. This may allow, for example, minimally trained users (including general practitioners, paramedics, and even patients) to obtain accurate anatomical measurements, with minimal time investment and with high confidence in the results.

This automatic measurement point detection system disclosed herein has particular, but not exclusive, utility for measuring anatomy in ultrasound procedures such as prenatal exams, lung exams, etc. The automatic measurement point detection system detects features in the individual frames of the video, automatically places measurement points on the video frame, performs calculations based on the measurement points, generates anatomical measurements based on the calculations, and displays the measurements on the ultrasound console display, all in real time (e.g., at refresh rates as high as 60-100 Hz, so that the system has completed the measurements and annotations for one frame of the live video stream before the next frame is displayed).

The automatic measurement point detection system uses artificial intelligence (AI) deep learning (DL) or other machine learning (ML) technology. The AI model is trained using ultrasound video frames that show a particular anatomy (e.g., a fetal head, abdomen, or femur), and that are human-annotated with measurement points (calipers). Once the AI model is trained, it can be incorporated into the ultrasound console's software to automatically identify and measure the imaged anatomy in real time. The clinician or other user simply moves and/or reorients the ultrasound probe over the body part containing the anatomy of interest, and the trained AI model identifies the anatomy and computes positions for the measurement points in real time (e.g., identifies the locations for the measurement points (e.g., the coordinates of the pixel(s) making up the measurement points among the pixels forming the anatomical image frame)), to automatically calculate the desired anatomical measurement. No intervention from the user is required, other than acquiring the relevant ultrasound images. The automatic measurement point detection system can complete hundreds of measurements at real time frame rates, which are analyzed for convergence and plane alignment. Once measurement convergence is identified (e.g., through the detection of maximum anatomy length or ellipse symmetry), the user interface indicates the most accurate measurement has been achieved, at which point the examination may be complete, or the user may move on to another anatomical feature for which measurements are desired.

The main elements of the automatic measurement point detection system include: (1) An AI neural net model trained on both ultrasound anatomy and human-generated measurement points (or bounding boxes thereof). (2) An error filtering step to confirm the measurement points are within the detected anatomy box and are compatible. (For example, femur endpoints cannot be located in the head.) (3) A calculation engine to derive the desired anatomical measurements from the identified measurement points. (4) Tests to detect measurement convergence. For example, a distance measurement such as femur length may be considered complete when the maximum distance is achieved (e.g., several seconds have elapsed without a longer measurement being detected). For more complex measurements such as circumferences or areas, the system may check for ellipse symmetry, maximum circumference, maximum area, and/or the presence of certain anatomical features that indicate the proper measurement plane. (5) User interface graphics to display measurements and status.

The automatic measurement point detection system can be used with live and/or recorded ultrasound video streams. Example deep learning models have been trained for measuring fetal femur length, head circumference and abdominal circumference, and models can also be trained using live or recorded video from a medical imaging device using other imaging modalities, including but not limited to visible light, angiography/fluoroscopy (X-ray), computer-aided tomography (CAT) scan, magnetic resonance imaging (MRI), intravascular ultrasound (IVUS), etc.

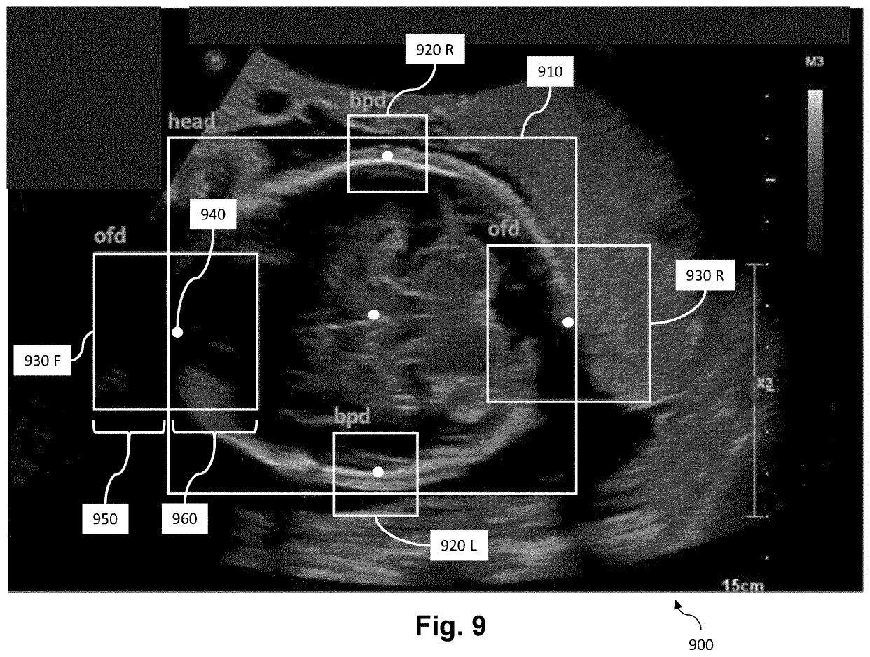

FIGS. 9, 16, and 19, below, are example images passed to the AI training step with fetal anatomy and measurement endpoints shown with bounding boxes. The neural net is “trained” by feeding a number (e.g., tens, hundreds or thousands) of images with annotated with anatomy identifications and measurement points. As the training process progresses, the neural net model “learns” the anatomy and measurement point positions and can accurately predict this information on new ultrasound images.

One distinct advantage of training with human-annotated anatomy and measurement endpoints is the ability to verify the results and filter potential errors. When detection data is received from the AI model, filtering rules are applied. For example, there must be a detection of the anatomy box itself (e.g., head, abdomen, femur) as well as the measurement points, and furthermore the measurement points must actually fall within the anatomy box.

### Examples: For the femur measurement to be completed, there must be two femur endpoints

located within the femur anatomy box. For the head circumference measurement to be completed, there must be two biparietal diameter (BPD) and two occipitofrontal diameter (OFD) endpoints located in the head anatomy box AND we must detect the thalamus structure in the head to identify the correct plane position. These rules ensure the neural network calculates the measurement at the precise imaging plane. Other filtering rules may be used instead or in addition.

In one aspect, the user starts an exam on the ultrasound system and begins scanning the patient. As anatomy is visualized, measurements for that anatomy are automatically displayed and updated in real time. As the user moves the ultrasound probe in and out of the imaging plane, the measurement stabilizes, and the user is presented with a final result. For anatomy requiring area measurements, multiple components of the measurement may be displayed, such as length and width, in real time with an alignment graphic indicating plane alignment based on factors such as the ratio of length and width, the appearance of reference anatomy in the desired imaging plane, etc.

In another aspect, automatic measurement point detection system can be configured to provide single measurements. When the user visualizes the desired anatomy and freezes the image, the single measurement is automatically displayed and is editable as in traditional workflow.

The present disclosure aids substantially in obtaining anatomical measurements from live or recorded radiology videos, by automatically placing measurement points and performing measurement calculations, without the need for human intervention (e.g., without the need for a clinician to pause the live video stream to place measurement points and compute measurements). Implemented on a processor in communication with an ultrasound probe, the automatic measurement point detection system disclosed herein provides practical improvements in the ability of untrained or inexperienced clinicians to obtain accurate anatomical measurements of a radiology video. This improved anatomy measurement transforms a subjective, time-intensive process that is heavily reliant on professional experience into one that is objective and repeatable, without the normally routine need to train clinicians such as emergency department personnel to recognize particular anatomy in the video stream and perform accurate measurements. This unconventional approach improves the functioning of the ultrasound imaging system, by providing reliable measurement of anatomical features or pathologies.

The automatic measurement point detection system may be implemented as a process at least partially viewable on a display, and operated by a control process executing on a processor that accepts user inputs from a keyboard, mouse, or touchscreen interface, and that is in communication with one or more sensor probes. In that regard, the control process performs certain specific operations in response to different inputs or selections made at different times. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the automatic measurement point detection system. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. For the sake of brevity, however, the numerous iterations of these combinations will not be described separately.

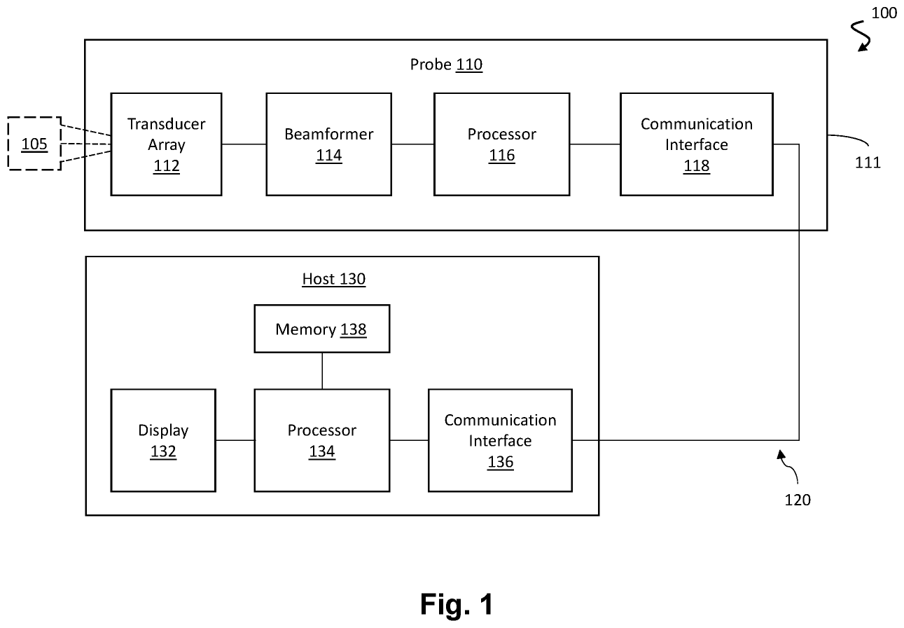

FIG. 1 is a schematic, diagrammatic representation of an ultrasound imaging system 100, according to aspects of the present disclosure. The ultrasound imaging system 100 may for example be used to acquire ultrasound video clips that may be used to train the automatic measurement point detection system, or that may be analyzed and highlighted in a clinical setting (whether in real time, near-real time, or as post-processing of stored video clips) by the automatic measurement point detection system.

The ultrasound imaging system 100 is used for scanning an area or volume of a subject's body. A subject may include a patient of an ultrasound imaging procedure, or any other person, or any suitable living or non-living organism or structure. The ultrasound imaging system 100 includes an ultrasound imaging probe 110 in communication with a host 130 over a communication interface or link 120. The probe 110 may include a transducer array 112, a beamformer 114, a processor circuit 116, and a communication interface 118. The host 130 may include a display 132, a processor circuit 134, a communication interface 136, and a memory 138 storing subject information.

In some aspects, the probe 110 is an external ultrasound imaging device including a housing 111 configured for handheld operation by a user. The transducer array 112 can be configured to obtain ultrasound data while the user grasps the housing 111 of the probe 110 such that the transducer array 112 is positioned adjacent to or in contact with a subject's skin. The probe 110 is configured to obtain ultrasound data of anatomy within the subject's body while the probe 110 is positioned outside of the subject's body for general imaging, such as for abdomen imaging, liver imaging, etc. In some aspects, the probe 110 can be an external ultrasound probe, a transthoracic probe, and/or a curved array probe.

In other aspects, the probe 110 can be an internal ultrasound imaging device and may comprise a housing 111 configured to be positioned within a lumen of a subject's body for general imaging, such as for abdomen imaging, liver imaging, etc., In some aspects, the probe 110 may be a curved array probe. Probe 110 may be of any suitable form for any suitable ultrasound imaging application including both external and internal ultrasound imaging.

In some aspects, aspects of the present disclosure can be implemented with medical images of subjects obtained using any suitable medical imaging device and/or modality. Examples of medical images and medical imaging devices include x-ray images (angiographic images, fluoroscopic images, images with or without contrast) obtained by a medical imaging device such as an ultrasound imaging device, X-ray imaging device, computed tomography (CT) images obtained by a CT imaging device, positron emission tomography-computed tomography (PET-CT) images obtained by a PET-CT imaging device, magnetic resonance images (MRI) obtained by an MRI imaging device, single-photon emission computed tomography (SPECT) images obtained by a SPECT imaging device, optical coherence tomography (OCT) images obtained by an OCT imaging device, and intravascular photoacoustic (IVPA) images obtained by an IVPA imaging device. The medical imaging device can obtain the medical images while positioned outside the subject body, spaced from the subject body, adjacent to the subject body, in contact with the subject body, and/or inside the subject body.

For an ultrasound imaging device, the transducer array 112 emits ultrasound signals towards an anatomical object 105 of a subject and receives echo signals reflected from the object 105 back to the transducer array 112. The ultrasound transducer array 112 can include any suitable number of acoustic elements, including one or more acoustic elements and/or a plurality of acoustic elements. In some instances, the transducer array 112 includes a single acoustic element. In some instances, the transducer array 112 may include an array of acoustic elements with any number of acoustic elements in any suitable configuration. For example, the transducer array 112 can include between 1 acoustic element and 10000 acoustic elements, including values such as 2 acoustic elements, 4 acoustic elements, 36 acoustic elements, 64 acoustic elements, 128 acoustic elements, 500 acoustic elements, 812 acoustic elements, 1000 acoustic elements, 3000 acoustic elements, 8000 acoustic elements, and/or other values both larger and smaller. In some instances, the transducer array 112 may include an array of acoustic elements with any number of acoustic elements in any suitable configuration, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of acoustic elements (e.g., one or more rows, one or more columns, and/or one or more orientations) can be uniformly or independently controlled and activated. The transducer array 112 can be configured to obtain one-dimensional, two-dimensional, and/or three-dimensional images of a subject's anatomy. In some aspects, the transducer array 112 may include a piezoelectric micromachined ultrasound transducer (PMUT), capacitive micromachined ultrasonic transducer (CMUT), single crystal, lead zirconate titanate (PZT), PZT composite, other suitable transducer types, and/or combinations thereof.

The object 105 may include any anatomy or anatomical feature, such as a kidney, liver, and/or any other anatomy of a subject. The present disclosure can be implemented in the context of any number of anatomical locations and tissue types, including without limitation, organs including the liver, kidneys, gall bladder, pancreas, lungs; ducts; intestines; nervous system structures including the brain, dural sac, spinal cord and peripheral nerves; the urinary tract; as well as valves within the blood vessels, blood, abdominal organs, and/or other systems of the body. In some aspects, the object 105 may include malignancies such as tumors, cysts, lesions, hemorrhages, or blood pools within any part of human anatomy. The anatomy may be a blood vessel, as an artery or a vein of a subject's vascular system, including cardiac vasculature, peripheral vasculature, neural vasculature, renal vasculature, and/or any other suitable lumen inside the body. In addition to natural structures, the present disclosure can be implemented in the context of man-made structures such as, but without limitation, heart valves, stents, shunts, filters, implants and other devices.

The beamformer 114 is coupled to the transducer array 112. The beamformer 114 controls the transducer array 112, for example, for transmission of the ultrasound signals and reception of the ultrasound echo signals. In some aspects, the beamformer 114 may apply a time-delay to signals sent to individual acoustic transducers within an array in the transducer 112 such that an acoustic signal is steered in any suitable direction propagating away from the probe 110. The beamformer 114 may further provide image signals to the processor circuit 116 based on the response of the received ultrasound echo signals. The beamformer 114 may include multiple stages of beamforming. The beamforming can reduce the number of signal lines for coupling to the processor circuit 116. In some aspects, the transducer array 112 in combination with the beamformer 114 may be referred to as an ultrasound imaging component.

The processor 116 is coupled to the beamformer 114. The processor 116 may also be described as a processor circuit, which can include other components in communication with the processor 116, such as a memory, beamformer 114, communication interface 118, and/or other suitable components. The processor 116 may include a central processing unit (CPU), a graphical processing unit (GPU), a digital signal processor (DSP), an application specific integrated circuit (ASIC), a controller, a field programmable gate array (FPGA) device, another hardware device, a firmware device, or any combination thereof configured to perform the operations described herein. The processor 116 may also be implemented as a combination of computing devices, e.g., a combination of a DSP and a microprocessor, a plurality of microprocessors, one or more microprocessors in conjunction with a DSP core, or any other such configuration. The processor 116 is configured to process the beamformed image signals. For example, the processor 116 may perform filtering and/or quadrature demodulation to condition the image signals. The processor 116 and/or 134 can be configured to control the array 112 to obtain ultrasound data associated with the object 105.

The communication interface 118 is coupled to the processor 116. The communication interface 118 may include one or more transmitters, one or more receivers, one or more transceivers, and/or circuitry for transmitting and/or receiving communication signals. The communication interface 118 can include hardware components and/or software components implementing a particular communication protocol suitable for transporting signals over the communication link 120 to the host 130. The communication interface 118 can be referred to as a communication device or a communication interface module.

The communication link 120 may be any suitable communication link. For example, the communication link 120 may be a wired link, such as a universal serial bus (USB) link or an Ethernet link. Alternatively, the communication link 120 may be a wireless link, such as an ultra-wideband (UWB) link, an Institute of Electrical and Electronics Engineers (IEEE) 802.11 WiFi link, or a Bluetooth link.

At the host 130, the communication interface 136 may receive the image signals. The communication interface 136 may be substantially similar to the communication interface 118. The host 130 may be any suitable computing and display device, such as a workstation, a personal computer (PC), a laptop, a tablet, or a mobile phone.

The processor 134 is coupled to the communication interface 136. The processor 134 may also be described as a processor circuit, which can include other components in communication with the processor 134, such as the memory 138, the communication interface 136, and/or other suitable components. The processor 134 may be implemented as a combination of software components and hardware components. The processor 134 may include a central processing unit (CPU), a graphics processing unit (GPU), a digital signal processor (DSP), an application-specific integrated circuit (ASIC), a controller, an FPGA device, another hardware device, a firmware device, or any combination thereof configured to perform the operations described herein. The processor 134 may also be implemented as a combination of computing devices, e.g., a combination of a DSP and a microprocessor, a plurality of microprocessors, one or more microprocessors in conjunction with a DSP core, or any other such configuration. The processor 134 can be configured to generate image data from the image signals received from the probe 110. The processor 134 can apply advanced signal processing and/or image processing techniques to the image signals. In some aspects, the processor 134 can form a three-dimensional (3D) volume image from the image data. In some aspects, the processor 134 can perform real-time processing on the image data to provide a streaming video of ultrasound images of the object 105. In some aspects, the host 130 includes a beamformer. For example, the processor 134 can be part of and/or otherwise in communication with such a beamformer. The beamformer in the in the host 130 can be a system beamformer or a main beamformer (providing one or more subsequent stages of beamforming), while the beamformer 114 is a probe beamformer or micro-beamformer (providing one or more initial stages of beamforming).

The memory 138 is coupled to the processor 134. The memory 138 may be any suitable storage device, such as a cache memory (e.g., a cache memory of the processor 134), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, solid state drives, other forms of volatile and non-volatile memory, or a combination of different types of memory.

The memory 138 can be configured to store subject information, measurements, data, or files relating to a subject's medical history, history of procedures performed, anatomical or biological features, characteristics, or medical conditions associated with a subject, computer readable instructions, such as code, software, or other application, as well as any other suitable information or data. The memory 138 may be located within the host 130. Subject information may include measurements, data, files, other forms of medical history, such as but not limited to ultrasound images, ultrasound videos, and/or any imaging information relating to the subject's anatomy. The subject information may include parameters related to an imaging procedure such as an anatomical scan window, a probe orientation, and/or the subject position during an imaging procedure. The memory 138 can also be configured to store information related to the training and implementation of machine learning algorithms (e.g., neural networks) and/or information related to implementing image recognition algorithms for detecting/segmenting anatomy, image quantification algorithms, and/or image acquisition guidance algorithms, including those described herein.

The display 132 is coupled to the processor circuit 134. The display 132 may be a monitor or any suitable display. The display 132 is configured to display the ultrasound images, image videos, and/or any imaging information of the object 105.

The ultrasound imaging system 100 may be used to assist a sonographer in performing an ultrasound scan. The scan may be performed in a at a point-of-care setting. In some instances, the host 130 is a console or movable cart. In some instances, the host 130 may be a mobile device, such as a tablet, a mobile phone, or portable computer. During an imaging procedure, the ultrasound system can acquire an ultrasound image of a particular region of interest within a subject's anatomy. The ultrasound imaging system 100 may then analyze the ultrasound image to identify various parameters associated with the acquisition of the image such as the scan window, the probe orientation, the subject position, and/or other parameters. The ultrasound imaging system 100 may then store the image and these associated parameters in the memory 138. At a subsequent imaging procedure, the ultrasound imaging system 100 may retrieve the previously acquired ultrasound image and associated parameters for display to a user which may be used to guide the user of the ultrasound imaging system 100 to use the same or similar parameters in the subsequent imaging procedure, as will be described in more detail hereafter.

In some aspects, the processor 134 may utilize deep learning-based prediction networks to identify parameters of an ultrasound image, including an anatomical scan window, probe orientation, subject position, and/or other parameters. In some aspects, the processor 134 may receive metrics or perform various calculations relating to the region of interest imaged or the subject's physiological state during an imaging procedure. These metrics and/or calculations may also be displayed to the sonographer or other user via the display 132.

Before continuing, it should be noted that the examples described above are provided for purposes of illustration, and are not intended to be limiting. Other devices and/or device configurations may be utilized to carry out the operations described herein.

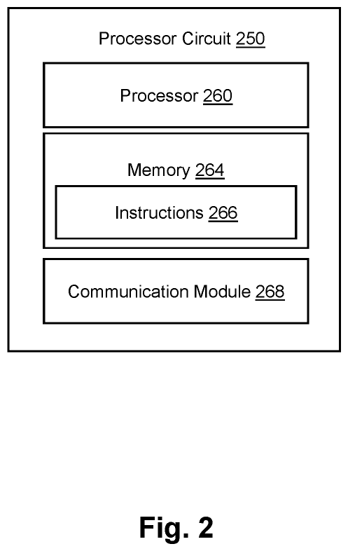

FIG. 2 is a schematic diagram of a processor circuit 250, according to aspects of the present disclosure. The processor circuit 250 may be implemented in the ultrasound imaging system 100, or other devices or workstations (e.g., third-party workstations, network routers, etc.), or on a cloud processor or other remote processing unit, as necessary to implement the method. As shown, the processor circuit 250 may include a processor 260, a memory 264, and a communication module 268. These elements may be in direct or indirect communication with each other, for example via one or more buses.

The processor 260 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 260 may also comprise another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 264 includes a non-transitory computer-readable medium. The memory 264 may store instructions 266. The instructions 266 may include instructions that, when executed by the processor 260, cause the processor 260 to perform the operations described herein. Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 250, and other processors or devices. In that regard, the communication module 268 can be an input/output (I/O) device. In some instances, the communication module 268 facilitates direct or indirect communication between various elements of the processor circuit 250 and/or the ultrasound imaging system 100. The communication module 268 may communicate within the processor circuit 250 through numerous methods or protocols. Serial communication protocols may include but are not limited to United States Serial Protocol Interface (US SPI), Inter-Integrated Circuit (I2C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, model sharing between the processor and central server, or readings from the ultrasound imaging system 100) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

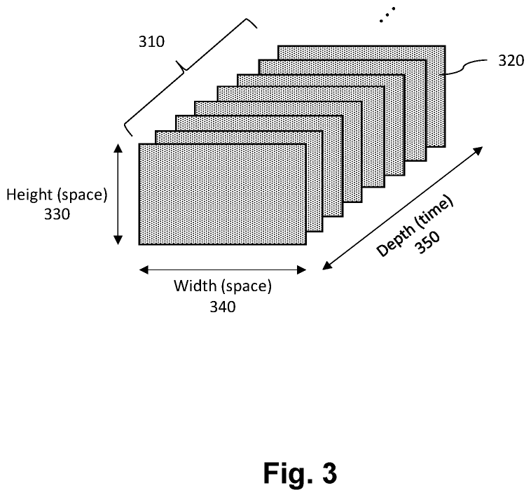

FIG. 3 is a schematic, diagrammatic representation of a radiology (e.g., ultrasound) video stream 310 (whether live or recorded), according to aspects of the present disclosure. The ultrasound video stream 310 includes a number of frames 320. In an example, the ultrasound video stream 310 is captured at a frame rate of 30, 60, or 100 frames per second. Each frame as a Y-axis or height 330, and X-axis or width 340, which are spatial dimensions representing a 2D cross-section of the objects being imaged by the ultrasound imaging system. In addition, the ultrasound video stream 310 includes a depth or time axis 350, representing the times at which each frame 320 of the video stream 310 was captured. Thus, the ultrasound video stream 310 may be considered a 3D data structure. The video stream 310 can be any suitable modality with 2D image frames over time, such as x-ray, MRI, CT, etc.

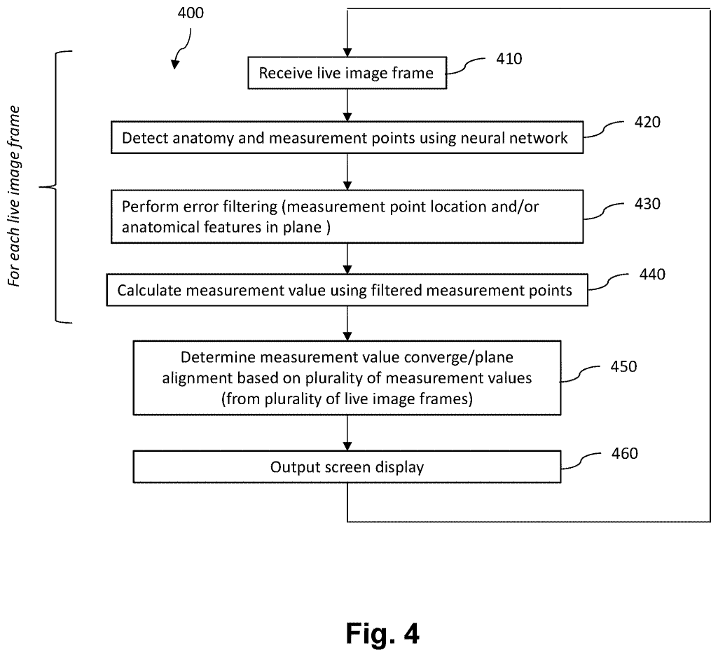

FIG. 4 is a schematic, diagrammatic representation, in flow diagram form, of an example automatic measurement point detection method 400, according to aspects of the present disclosure. It is understood that the steps of method 400 may be performed in a different order than shown in FIG. 4, additional steps can be provided before, during, and after the steps, and/or some of the steps described can be replaced or eliminated in other aspects. One or more of steps of the method 400 can be carried by one or more devices and/or systems described herein, such as components of the ultrasound system 100 and/or processor circuit 250.

In step 410, the method 400 includes receiving an image frame from the live or recorded ultrasound video stream (or other radiology video stream).

In step 420, the method 400 includes detecting/identifying anatomy (e.g., a fetal head, abdomen, or femur) with a neural network, and placing measurement points on the image frame in relation to the anatomy. Depending on the implementation, the measurement points may be placed by the same neural network that detects the anatomy, or by a different neural network.

Anatomy detection based on machine learning (ML) or other neural network (NN) based artificial intelligence (AI) may for example be through object detection or bounding box detection, classification, segmentation, or other related means. Examples of classification networks can be found for example in U.S. Provisional Patent Application No. 63/293,232, filed Dec. 23, 2021, entitled “Methods and systems for clinical scoring a lung ultrasound” and U.S. Provisional Patent Application No. 63/294,501, filed Dec. 29, 2021, entitled “Machine-learning image processing independent of reconstruction filter”, each of which is incorporated by reference as though fully set forth herein. An example of bounding box object detection can be found in India patent application No. 202141034243, filed Jul. 29, 2021, entitled “Generating location data” (International Application No. PCT/EP2022/070410), which are incorporated by reference as though fully set forth herein. Examples of image or video segmentation can be found for example in U.S. Provisional Patent Application No. 63/325,660, filed Mar. 31, 2022, entitled “Methods and systems for ultrasound-based structure localization using image and user inputs”, and U.S. Publication No. 2022/0198669, entitled “Segmentation and view guidance in ultrasound imaging and associated devices, systems, and methods”, each of which is incorporated by reference as though fully set forth herein.

In step 430, the method 400 includes filtering the anatomy detection and measurement points for reasonableness. For example, an anatomy detection may be considered valid if certain related anatomical features can be identified in the image frame, or if the eccentricity of elliptical features falls within a certain specified range, and may otherwise be considered invalid. In some aspects, and invalid anatomy detection may automatically invalidate any measurement points placed in reference to that anatomy. A measurement point may be considered valid if it falls within a bounding box of the valid detected anatomy, or within specific regions of the detected anatomy. For example, a femur endpoint may be considered invalid if it occurs outside the femur bounding box, or near the middle of the femur rather than near one of the ends. Such filtering provides a check and balance against spurious predictions by the neural network(s), so that the system is configured to generate the first measurement value based on the identification of the anatomy.

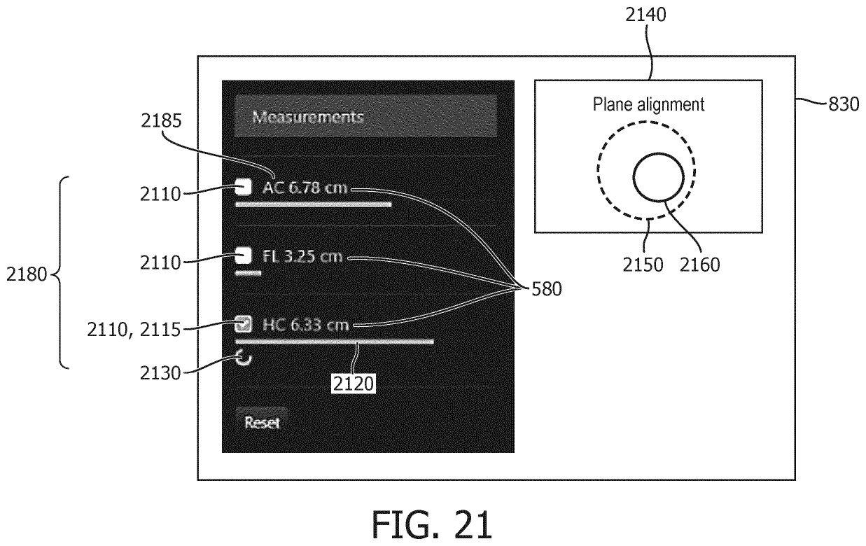

In step 440, the method 400 includes calculating one or more measurement values using the filtered measurement points. Measurements may for example include the length, width, area, diameter, or circumference of the identified anatomy, or other anatomical measurements. A measurement could for example be made using the measurement points directly (e.g., the length/distance between two measurement points). For example, the femur length can be the length/distance between the femur endpoints. In other cases, a measurement may combine multiple measurements values. For example, the head circumference can be a combination of the biparietal diameter (BPD, a first length/distance between two measurement points) and the occipitofrontal diameter (AFD, a second length/distance between two measurement points). E.g., HC=1.62×(BPD+OFD). Similarly, the abdominal circumference (AC) may be obtained from the anterior-posterior abdominal diameter (APAD) and transverse abdominal diameter (TAD) by AC=π(APAD+TAD)/2=1.57 (APAD+TAD). A displayed measurement value could be a numerical value of a measurement itself, or it could be a derived value calculated using first measurement value. For example, OFD and BPD can be obtained as direct measurements (e.g., distances), whereas HC is a derived value that is calculated using the OFD and BPD. The displayed measurement could also be a visual depiction of the measurement points (without or without a line/curve between the points), or could be or include the convergence progress indicators discussed in FIGS. 21-23. Measurement values may be two or more different measurements of the same anatomical features. Two or more consecutive measurement values of the same anatomical feature could be the same or different from one another. The values could for example be different because different image frames show different portions of the same anatomy (e.g., different image plane), not because a different anatomical feature is being measured.

In an example, one or several metrics are calculated from the detections. The metrics may be hard-coded into the system, or may be selectable in real time or near-real time to represent clinically relevant parameters derived from the detections. For example, in a screening/triage context, the operator may be interested in picking up features of any size, as long as they have been detected with sufficient confidence. In this setting, a metric defined as the maximum confidence of all detections (but independent of detection area) of a feature type may be appropriate. Alternatively, in a diagnostic context the operator may already know that very small findings are not clinically significant but that larger findings may indicate pathology. Therefore, a metric may be defined as the maximum area of all detection bounding boxes, or the maximum of a product of area and confidence of all detections. This way, the metric would not be sensitive to small findings (even those with high confidence).

In step 450, the method 400 includes determining convergence of the measurement value(s) and/or plane alignment of the image frame relative to the anatomy. For example, as different measurement values are calculated at each cycle of the method (e.g., each image frame of the ultrasound video stream), the method may include keeping track of the largest value for the measurement, and may consider the measurement “converged” when a certain time or a certain number of frames have elapsed without a larger value being detected, or “non-converged” if the largest value is still changing, and thus the user needs to keep acquiring live ultrasound video to improve the measurements. In some aspects, the method uses cues such as the eccentricity of ellipses or the presence of specific anatomical features to determine whether the image frame has been captured at the desired location, alignment, or depth, and may consider the measurement “not converged” if the image frames containing the maximum values are improperly placed or aligned. Depending on the implementation, guidance may be provided to the user to move or reorient the ultrasound probe to obtain a better measurement value, or the user interface may simply indicate that the measurement value has not yet converged-a cue to the user to keep moving the ultrasound probe.

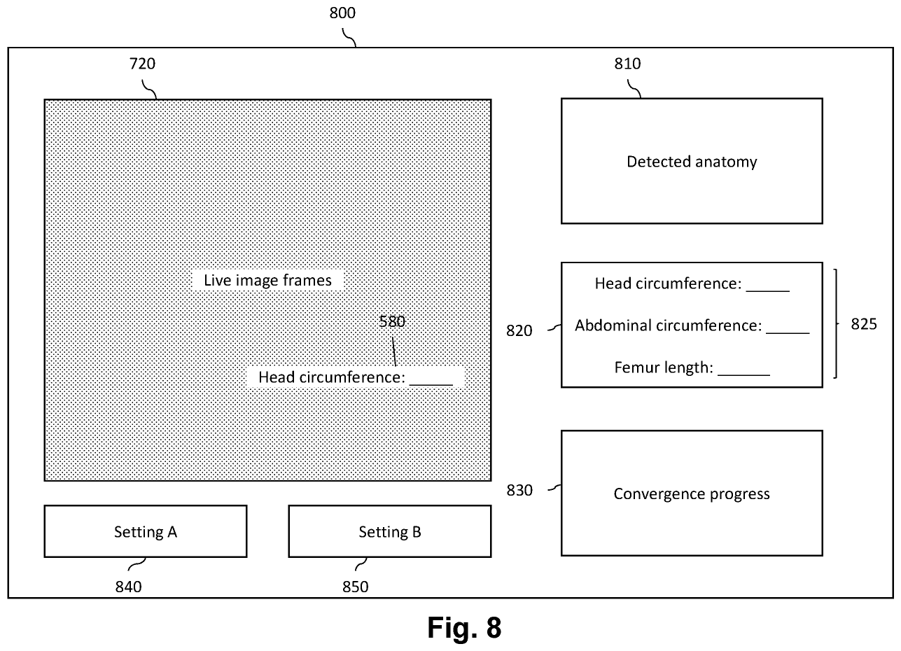

In step 460, the method 400 includes displaying the measurement to the user (e.g., adjacent to or superimposed upon the current image frame of the video stream). Non-limiting example screen displays may be found below in FIGS. 8-23. Since the method can execute in real time, execution may then returns to step 410 to await receipt of the next image frame in the video stream).

Flow diagrams are provided herein for exemplary purposes; a person of ordinary skill in the art will recognize myriad variations that nonetheless fall within the scope of the present disclosure. For example, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide each of the steps described herein into a plurality of machine instructions, and may execute these instructions at the rate of several hundred, several thousand, several million, or several billion per second, in a single processor or across a plurality of processors. Such rapid execution may be necessary in order to execute the method in real time or near-real time as described herein. For example, in order to measure anatomical features in a real-time ultrasound video stream, steps 410-440 may need to execute faster than the frame rate of the video (e.g., 30, 60, or 100 executions per second.

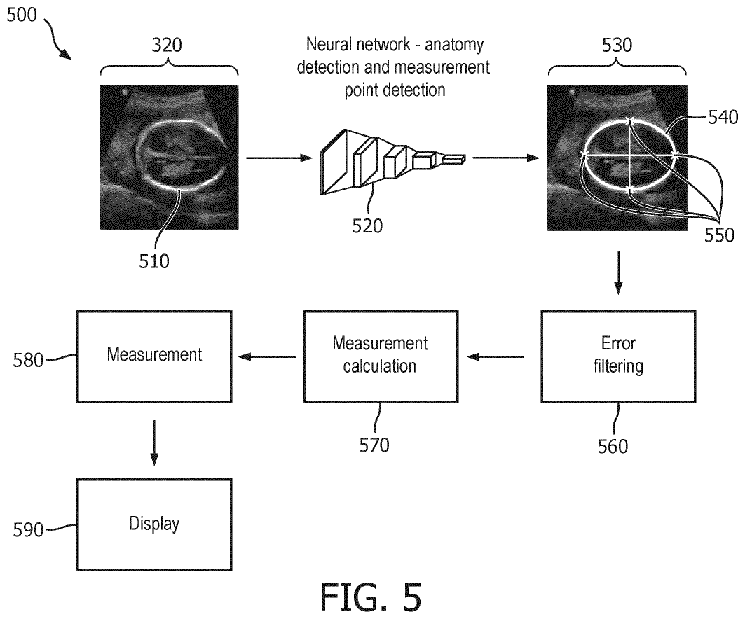

FIG. 5 is a schematic, diagrammatic representation, in block diagram form, of at least a portion of an automatic measurement point detection system, according to aspects of the present disclosure. An image frame 320 of the ultrasound video stream is received by an object detector/analyzer 520 (e.g., a deep learning network or other machine learning neural network or artificial intelligence), which performs feature detection (e.g., detecting specified anatomical features) on the image frame 320, and determines the locations of measurement points. In some aspects, the object detector/analyzer 520 comprises two separate neural networks (e.g., an object detector and an object analyzer), although a single neural network may be easier to train and more computationally efficient. The object detector/analyzer may output an annotated video frame 530 that marks the measured anatomical feature 540 and the measurement points 550.

The object detector/analyzer 520 may implement or include any suitable type of learning network. For example, in some aspects, the object detector/analyzer 520 could include a neural network, such as a convolutional neural network (CNN). In addition, the convolutional neural network may additionally or alternatively be or an encoder-decoder type network, or may utilize a backbone architecture based on other types of neural networks, such as an object detection network, classification network, etc. One example backbone network is the Darknet YOLO backbone, (e.g., Yolov3) which can be used for object detection. The CNN may for example include a set of N convolutional layers, where N may be any positive integer. Fully connected layers can be omitted when the CNN is a backbone. The CNN may also include max pooling layers and/or activation layers. Each convolutional layer may include a set of filters configured to extract features from an input (e.g., from the image frame 320). The value N and the size of the filters may vary depending on the aspects. In some instances, the convolutional layers may utilize any non-linear activation function, such as for example a leaky rectified non-linear (ReLU) activation function and/or batch normalization. The max pooling layers gradually shrink the high-dimensional output to a dimension of the desired result (e.g., a bounding box of a detected feature).

Fully connected layers may be referred to as perception or perceptive layers. In some aspects, perception/perceptive and/or fully connected layers may be found in the object detector/analyzer 520 (e.g., a multi-layer perceptron), to allow downstream processing (e.g., detection, segmentation, etc.). The object detector/analyzer 520 may also include max pooling layers and/or activation layers. The max pooling layers gradually shrink the high-dimensional output to a dimension of the desired result (e.g., a bounding box for a region of interest).

These descriptions are included for exemplary purposes; a person of ordinary skill in the art will appreciate that other types of AI learning models, with features similar to or dissimilar to those described above, may be used instead or in addition, without departing from the spirit of the present disclosure.

The annotated image frame 530, or the information used to generate the annotations, is then sent to an error filtering block 560, which filters the anatomy detection and measurement points for reasonableness as described above. For example, an anatomy detection may be considered valid if specified anatomical landmarks are identified in the image frame, or if the eccentricity of elliptical features falls within a certain specified range. A measurement point may be considered valid if it falls within the correct (and valid) anatomy.

The filtered measurement points are then sent to a measurement calculation block 570, which performs calculations such as determining the distance between two measurement points, the circumference or area of an ellipse defined by four measurement points, or otherwise.

The measurement calculation block produces one or more measurements 580, which are then shown on the display 590, along with the annotated image frame 530, the original image frame 320, the original image frame 320 with bounding boxes, or otherwise, as shown below.

Block diagrams are provided herein for exemplary purposes; a person of ordinary skill in the art will recognize myriad variations that nonetheless fall within the scope of the present disclosure. For example, block diagrams may show a particular arrangement of components, modules, services, steps, processes, or layers, resulting in a particular data flow. It is understood that some aspects of the systems disclosed herein may include additional components, that some components shown may be absent from some aspects, and that the arrangement of components may be different than shown, resulting in different data flows while still performing the methods described herein.

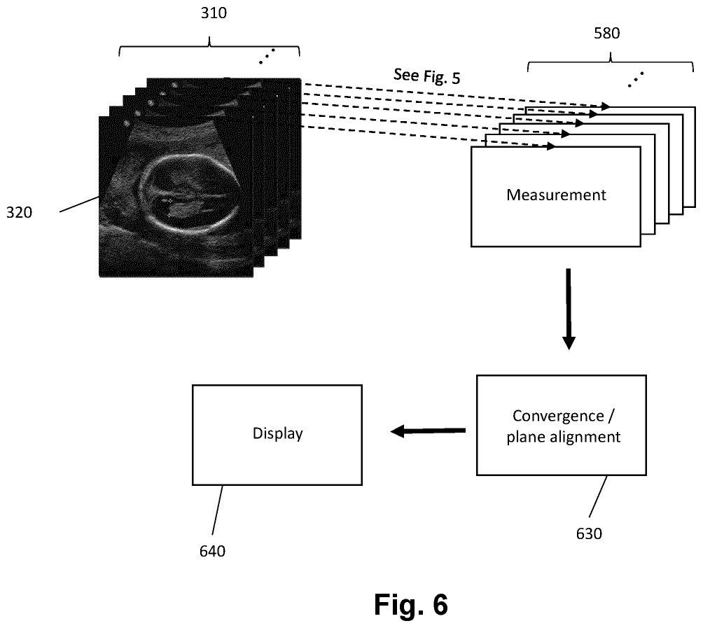

FIG. 6 is a schematic, diagrammatic representation, in block diagram form, of at least a portion of an automatic measurement point detection system, according to aspects of the present disclosure. Image frames 320 of the radiology video stream 310 yield a sequence of measurements 580 (e.g., one measurement per image frame 320). The measurements 580 are received by a convergence and plane alignment block 630, which determines convergence of the measurement value(s) and/or plane alignment of the image frame relative to the anatomy, as described above. For example, as different measurement values are calculated for each image frame, the convergence and plane alignment block 630 may keep track of the largest value for the measurement, and may use cues such as the eccentricity of ellipses or the presence of specific anatomical features to determine whether the image frame has been captured at the desired location, alignment, or depth.

The convergence and plane alignment block 630 then generates a graphical display 640 that can be overlaid on or displayed adjacent to the image frame. For example, the measurement may be textually or graphically shown as “not converged” or “in progress” if the image frames containing the maximum values are improperly placed or aligned, or if the maximum calculated value of the measurement is still increasing over time. Depending on the implementation, the graphical display 640 may also provide guidance to the user to move or reorient the ultrasound probe.

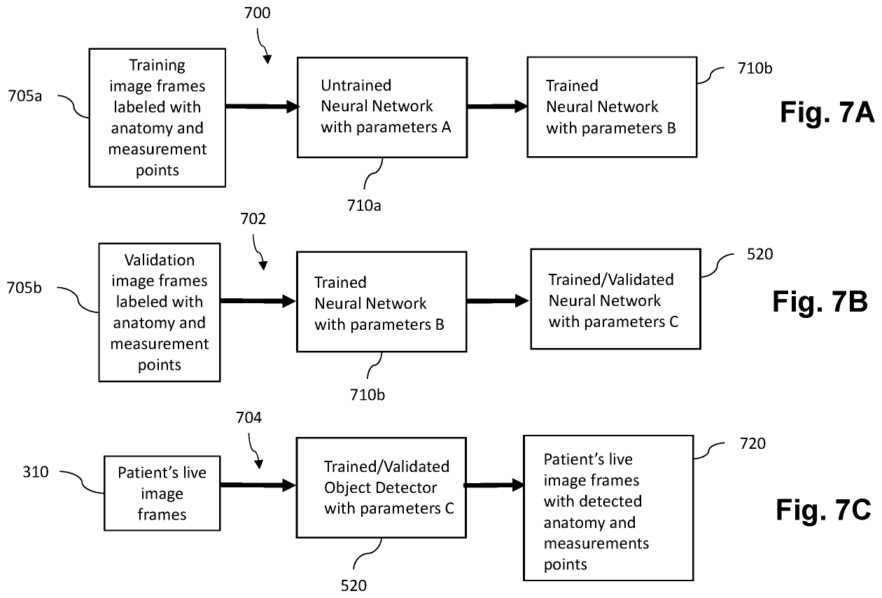

FIG. 7A is a schematic, diagrammatic overview, in block diagram form, of a training mode 700 for the object detector/analyzer 520, according to aspects of the present disclosure. In the example shown in FIG. 7A, a set of training data 705a that includes ultrasound video streams with hand-marked anatomy localizations and measurement points (e.g., surrounded by bounding boxes) is fed into an untrained object detector/analyzer 710a in an iterative training process that will be familiar to a person of ordinary skill in the art.

In particular, for object detection and measurement point placement using convolutional neural networks, large numbers of sample images are manually annotated by experts to delineate the localizations of features of interest. The parameters of a network model (e.g., the weights at each artificial neuron) are initialized with initial values A that may be random values or with results from training on prior datasets. In an iterative process, the network is used to make detection inferences on the training images, the results are compared with the ground truth annotations, and an optimizer is used to adjust the network parameters B until a metric of detection accuracy is maximized.

Thus, an output of this training process 700 is a trained object detector/analyzer 710b, wherein the parameters B (e.g., weights) are optimized for detection of the features in the training videos 705a.

FIG. 7B is a schematic, diagrammatic overview, in block diagram form, of a validation mode 702 for the object detector/analyzer 520, according to aspects of the present disclosure. In the validation mode, a set of hand-annotated validation videos (e.g., videos that include bounding boxes around any anatomy or measurement points identified by an expert, in each frame of each video) are fed into the trained object detector/analyzer 710b in order to determine whether human-identified features in the validation videos 705b are detected by the trained object detector/analyzer 710b to a desired level of accuracy.

In some cases, performance of the trained object detector/analyzer 710b may be deemed to be below the desired level of accuracy. In this case, the parameters B (e.g., weights) of the trained object detector/analyzer 710b may be adjusted until detection accuracy on the validation dataset (or the validation dataset plus the training dataset) reaches the desired accuracy. In such cases, an output of the validation process may be the trained object detector/analyzer 520, which may be identical to the trained object detector/analyzer 710b except for the adjusted parameters C (e.g., weights). In other cases, performance of the trained object detector/analyzer 710b may be deemed to be adequate, and so no adjustments to the parameters are made, and the trained object detector/analyzer 520 may be identical to (e.g., uses the same weights as) the trained object detector/analyzer 710b.

FIG. 7C is a schematic, diagrammatic overview, in block diagram form, of an inference mode or clinical usage mode 704 for the object detector/analyzer 520, according to aspects of the present disclosure. In clinical usage, an ultrasound video or video stream 310 is fed to the trained and validated object detector/analyzer 520 for analysis. In some cases, the video stream 310 may be acquired and analyzed in real time or near-real time. In other cases, the video stream may be retrieved from memory, storage, or a network. The trained and validated object detector/analyzer 520 then produces, as an output, an annotated version 720 of the video stream 310, which includes measurement points and detected anatomy.

Thus, in each frame of the video stream, the object detector/analyzer 520 is run to determine a localization and a confidence value for features (e.g., anatomy and measurement points). The localization can be determined in the form of a bounding box that tightly encloses the feature. Other forms of localizations are possible such as a binary mask indicating the image pixels that are part of the feature, or a polygon or other shape enclosing the feature. For any such localization, a center and an area of the detection can be determined. The confidence value can be determined as a normalized value in the range [0, 1], where 0 indicates lowest confidence, and 1 indicates highest confidence that the feature is present at that location.

The detection step can be based on conventional image processing including thresholding, filtering, and texture analysis, or can be based on machine learning, in particular using deep neural networks. One specific beneficial implementation of the detection is using a Yolo-type network such as Yolo3. An exemplary output of the detection step is, for each frame of the video stream, a list of detections for one or several types of features of interest. Each element in the detection list may for example include at least the confidence and area (and typically also the position, width, and height) of detection. For each frame i and feature f there is thus a list of detections {x,y,w,h; c}_{f,i}, where x,y represent the center coordinates, w,h represents the width and height, and c represents the confidence value of the detection. Other means of representing the detections may be used instead or in addition, without departing from the spirit of the present disclosure.

FIG. 8 is an example screen display 800 of the automatic measurement point detection system, according to aspects of the present disclosure. In the example shown in FIG. 8, the screen display 800 includes a location for the annotated image frames 720 with an overlaid measurement 580. The screen display 800 also includes a detected anatomy display area 810, a measurement reporting area 820 containing finalized measurements 825, a convergence progress indicator 830, and two control settings 840 and 850.

FIG. 9 is an example training image 900 for the automatic measurement point detection system, according to aspects of the present disclosure. In the example shown in FIG. 9, the training image 900 is of a fetal head, and includes a head bounding box 910, left and right biparietal (BPD) measurement point bounding boxes 920 L and 920 R, front and rear occipitofrontal diameter (OFD) measurement point bounding boxes 930F and 930R. Each bounding box includes a measurement point 940, as well as a region 950 that occurs predominantly outside the head, and a region 960 that occurs predominantly inside the head.

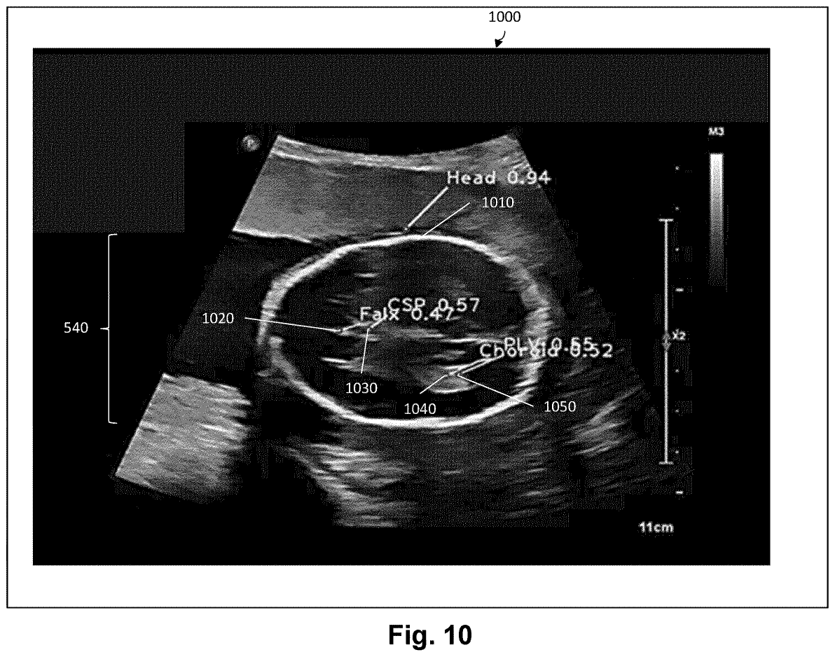

FIG. 10 is an example object detection image 1000 of the automatic measurement point detection system, according to aspects of the present disclosure. Visible are the head 1010, falx cerebri 1020, cavum septi pellucidi (CSP) 1030, choroid plexus 1040, and posterior lateral ventricle (PLV) 1050. These anatomical features are generally found in the imaging plane most suitable for making head circumference measurements, and may thus serve as indicators that the imaging plane of the ultrasound probe is properly positioned and aligned for the head circumference measurement. These particular anatomical landmarks are described here for exemplary purposes. Depending on the anatomy being measured, other landmarks may be used instead or in addition, or else the proper measurement plane may be detected based on geometry (e.g., the eccentricity of an ellipse), without reference to other anatomical landmarks.

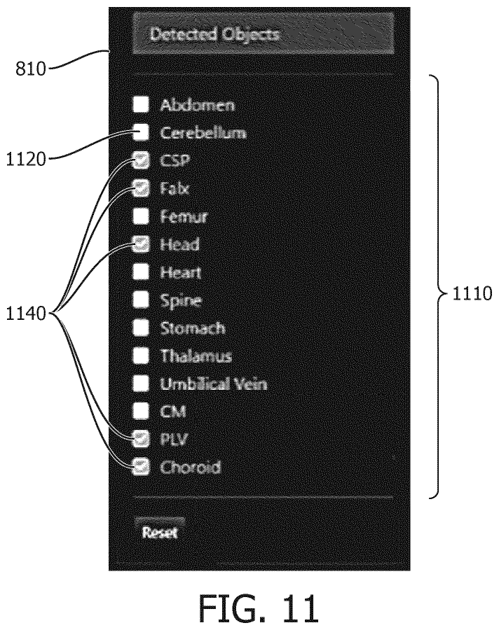

FIG. 11 is an example “detected objects” display 810 of the automatic measurement point detection system, according to aspects of the present disclosure. In the example shown in FIG. 11, the detected objects display 810 includes a number of anatomical features 1110, each with its own check box 1120. Checked boxes 1140 can indicate either anatomy that is currently visible in the ultrasound video stream, anatomy that has previously been detected, or anatomy whose detection has enabled an associated measurement to converge. In an example, an examination may be considered complete when all of the check boxes 1120 are checked.

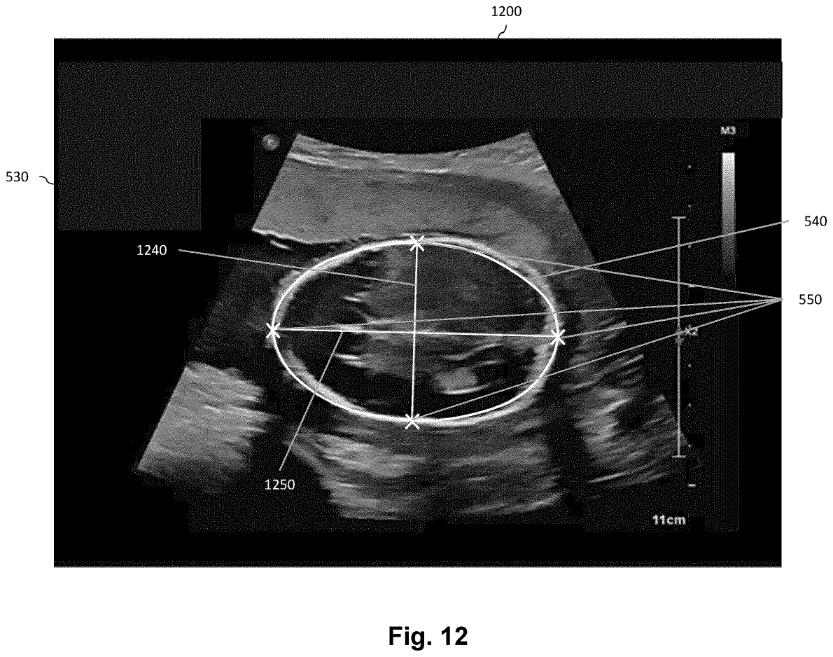

FIG. 12 is an example inference image 1200 generated by the automatic measurement point detection system, according to aspects of the present disclosure. The image includes a measured anatomical feature 540 (in this case, an ellipse representing the anatomy, which is a fetal head) as well as measurement points 550. Also visible are a biparietal diameter (BPD) line 1240 and an occipitofrontal diameter (OFD) line 1250. Depending on the implementation, these annotations may for example be displayed to the user along with the measurement values derived from them.

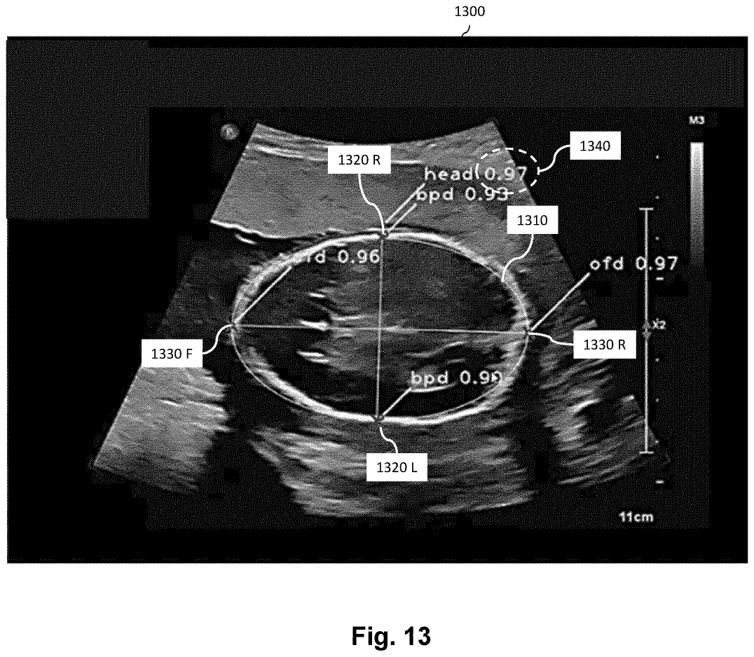

FIG. 13 is an example object detection image 1300 of the automatic measurement point detection system, according to aspects of the present disclosure. Visible are the detected anatomy 1310 and measurement points 1320L, 1320R, 1330 F, and 1330 R, as well as confidence values 1340 for the detections. Confidence thresholds may be a user-selectable value such that, for example, an anatomical feature is only shown if its detection confidence 1340 exceeds 50%, 80%, 90%, 95%, etc. Higher confidence thresholds may increase procedure times, as acquiring the anatomy may be more difficult and thus take longer. However, higher confidence thresholds may also improve the accuracy of the results.

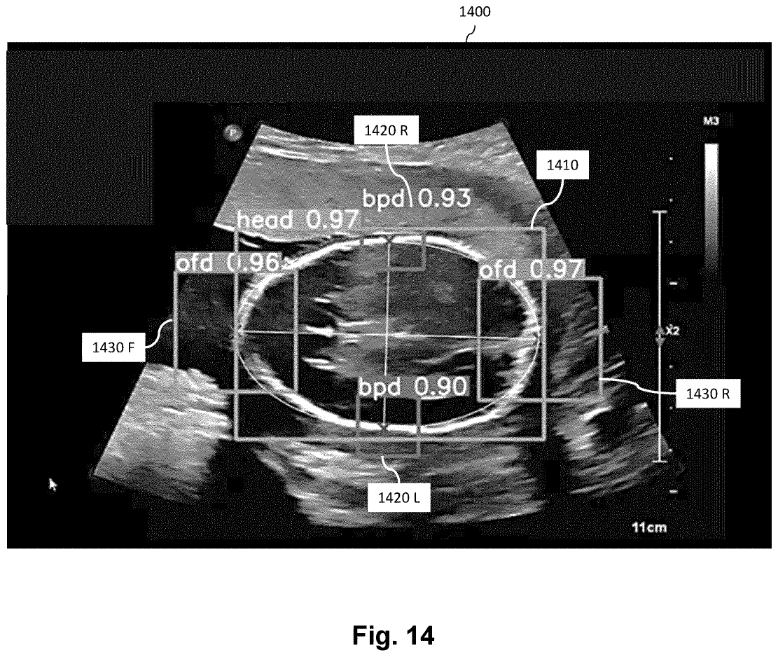

FIG. 14 is an example object detection image 1400 of the automatic measurement point detection system, according to aspects of the present disclosure. In this case, the detections are shown as bounding boxes 1410, 1420R, 1420L, 1430F and 1430R. Depending on the implementation, there may be a threshold associated with the center point of the measurement point box being inside the anatomy box. For example, a measurement point for the head may not necessarily have to be exactly inside the head (or the head anatomy box), but can be within a threshold distance (+/−) of the anatomy box.

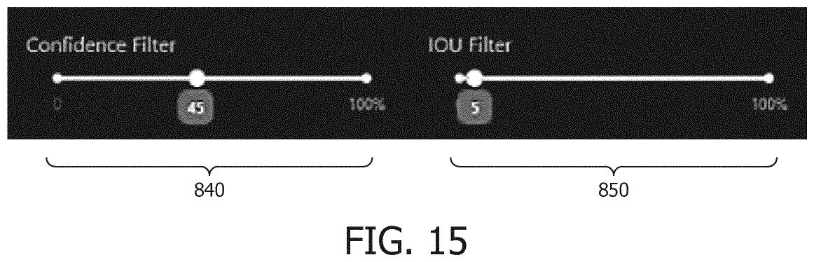

FIG. 15 is an example filter control set of the automatic measurement point detection system, according to aspects of the present disclosure. Visible are a confidence filter control 840 and an intersection over union (IOU) filter control 850. In an example, the confidence filter control 840 can be used to adjust (e.g., in real time) the confidence thresholds for anatomy detection. The IOU filter control can be used to set thresholds for weeding out duplicate detections. For example, if two femur endpoints are detected within a few millimeters of each other, that may be considered a single femur endpoint and thus a single measurement point, whereas if two femur endpoints are detected several centimeters apart, then they may be considered two different endpoints of the same femur.

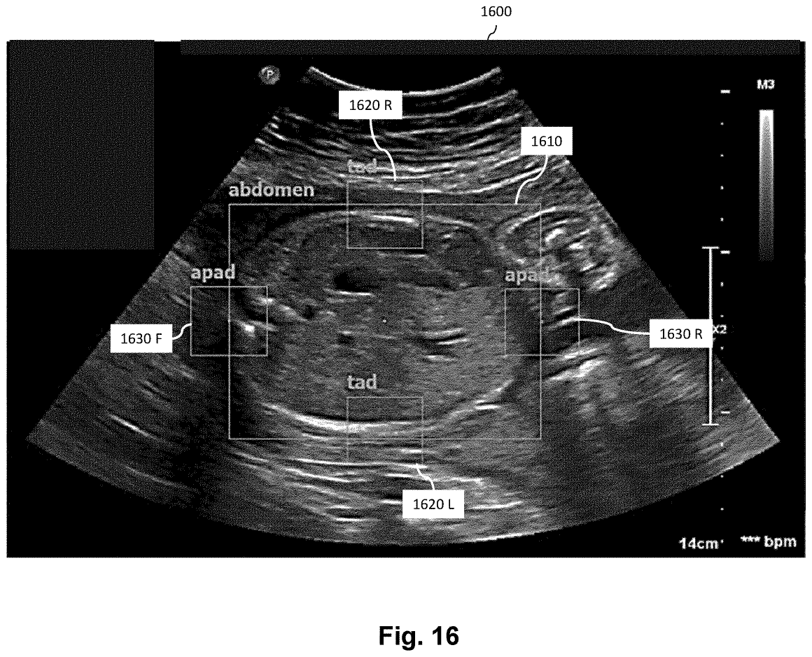

FIG. 16 is an example training image 1600 of the automatic measurement point detection system, according to aspects of the present disclosure. In the example shown in FIG. 16, the training image 1600 is of a fetal abdomen, and includes an abdomen bounding box 1610, left and right transverse abdominal diameter (TAD) measurement point bounding boxes 1620 L and 1620 R, front and rear anterior-posterior abdominal diameter (APAD) measurement point bounding boxes 1630F and 1630R.

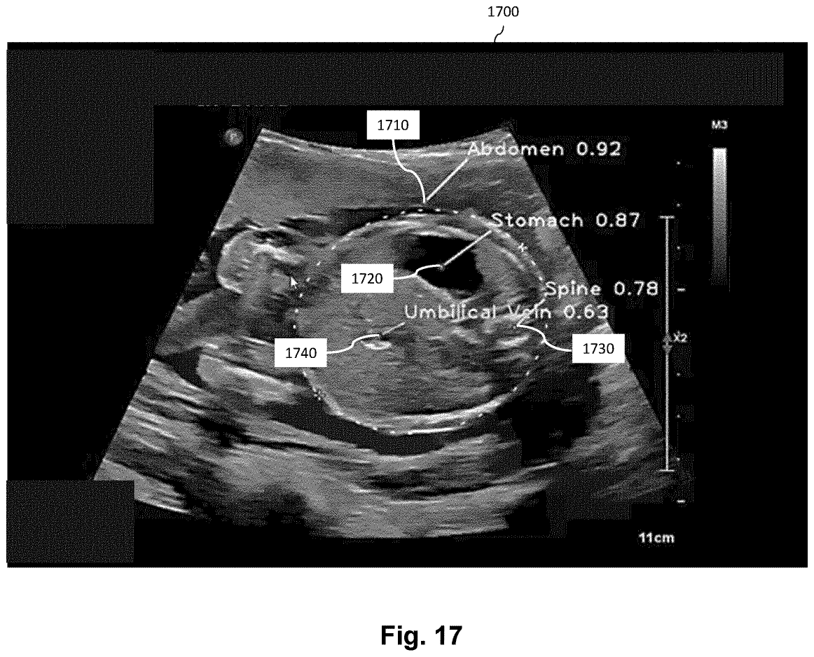

FIG. 17 is an example object detection image 1700 of the automatic measurement point detection system, according to aspects of the present disclosure. Visible are the abdomen 1710, stomach 1720, spine 1730, and umbilical vein 1740. In an example, the detection of these anatomical features may indicate that the imaging plane of the ultrasound probe is properly positioned and aligned for a measurement of abdominal diameter or circumference.

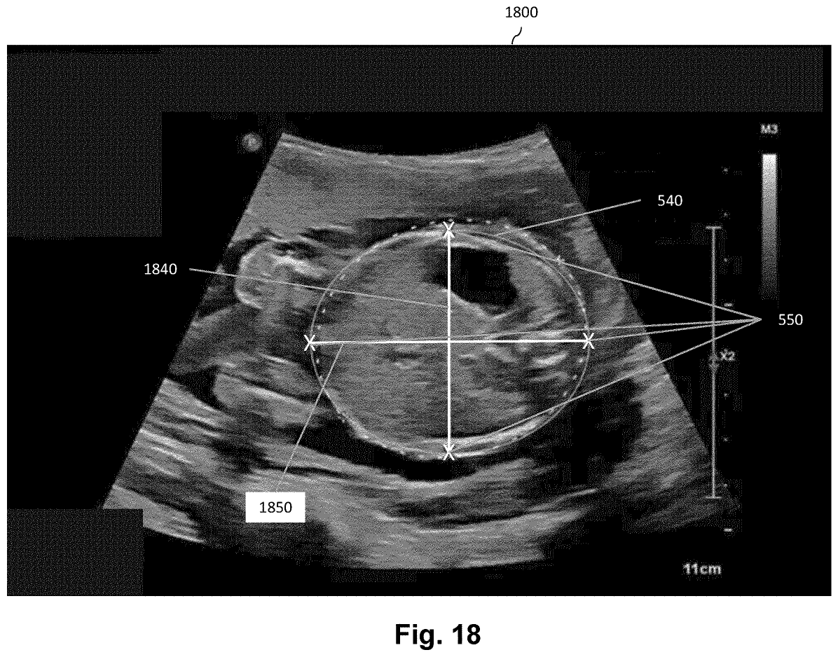

FIG. 18 is an example inference image 1800 generated by the automatic measurement point detection system, according to aspects of the present disclosure. The image includes the measured anatomical feature 540 (in this case, an ellipse representing the anatomy, which is a fetal abdomen) as well as measurement points 550. Also visible are an anterior-posterior abdominal diameter (APAD) line 1840 and a transverse abdominal diameter (TAD) line 1850. Depending on the implementation, these annotations may for example be displayed to the user along with the measurement values derived from them.

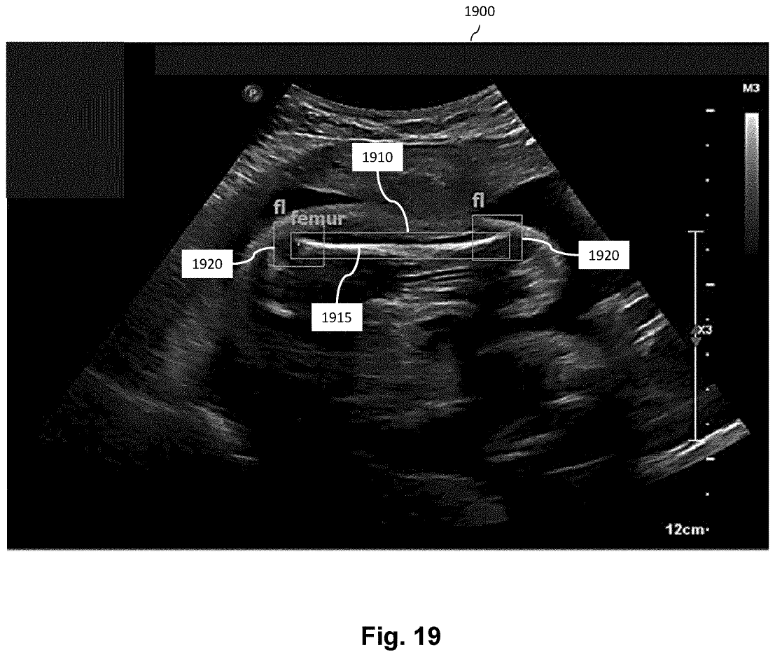

FIG. 19 is an example training image 1900 for the automatic measurement point detection system, according to aspects of the present disclosure. The image shows an anatomy bounding box 1910 of a fetal femur bone 1915, as well as bounding boxes 1920 for the endpoints of the femur bone 1915. The training image may be fed to the untrained neural network as described for example in FIG. 7A.

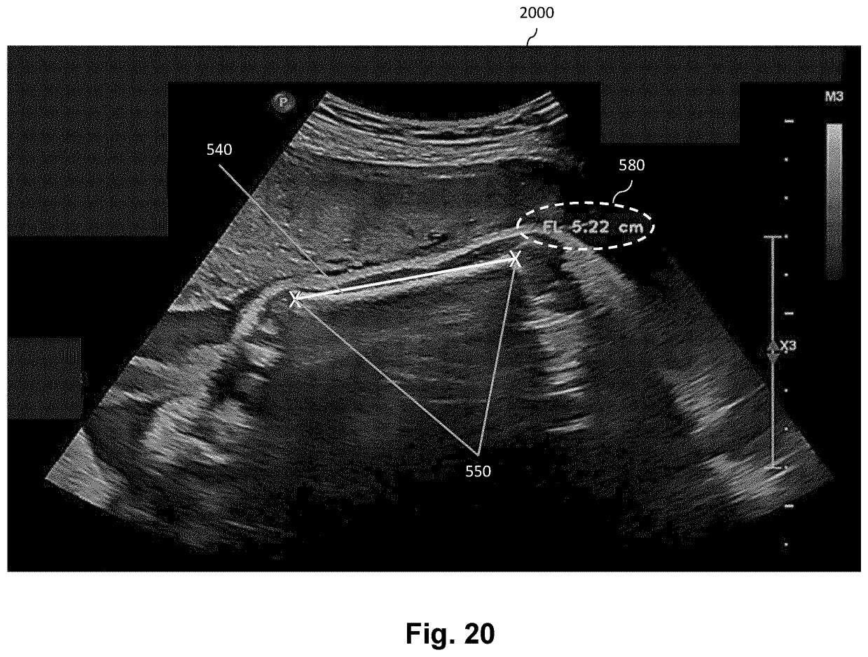

FIG. 20 is an example inference image 2000 generated by the automatic measurement point detection system, according to aspects of the present disclosure. The image includes the measured anatomical feature 540 (in this case, a straight line representing the anatomy, which is a fetal femur bone) as well as measurement points 550 indicating the endpoints of the femur. In this example, the measured anatomical feature 540 is simply the line connecting the two measurement points 550. Depending on the implementation, these annotations may for example be displayed to the user along with the measurement values derived from them.

Depending on the implementation, a measured anatomical feature 540 may be or include is the head circumference, BPD, OPD, femur length, abdominal circumference, TAD, APAD, or other anatomical feature of the patient.

FIG. 21 is an example convergence progress display 830 of the automatic measurement point detection system, according to aspects of the present disclosure. In the example shown in FIG. 21, the convergence progress display 830 includes a plurality of measurement values 580 and measurement labels 2185 for measurements 2180, each with a check box 2110, and a progress indicator 2120. A “waiting” or “in process” indicator 2130 and a checked checkbox 2115 are visible on one of the measurements 2180, indicating that this measurement 2180 is currently in progress and has not yet converged.

In an example, while a measurement 2180 is waiting to converge, every time a larger (or otherwise “more converged”) value is for that measurement 2180 is received, the measurement value 580 for that measurement 2180 is updated, and the progress indicator 2120 for that measurement 2180 gets longer. In some cases, the progress indicator 2120 may also change color so that, for example, when the measurement value 580 is changing rapidly, the corresponding progress indicator 2120 is red, and when the measurement value 580 is changing less frequently, the corresponding progress indicator 2120 is yellow, and when the measurement value 580 is no longer changing, the corresponding progress indicator 2120 is green. Thus, a green indicator 2120 for each measurement 2180 may indicate that all desired measurements have converged, and the examination is complete. In other instances, the color of the measurement value itself may change from red to yellow to green in the same way, or other visual indicators of convergence or non-convergence could be provided instead or in addition.

In the example shown in FIG. 21, the convergence progress display 830 also includes a plane alignment indicator 2140. The plane alignment indicator 2140 includes a desired imaging plane indicator 2150 and an actual imaging plane indicator 2160, in a bubble level configuration. The position and orientation of the actual imaging plane indicator 2160 relative to the desired imaging plane indicator may thus provide guidance to the clinician or other user to move or reorient the ultrasound imaging probe. For example, as the transducer is aligned, the small circle may center inside the larger circle. For example, alignment can be based on the intersection of the BPD and the OFD, or based on the intersection of the TAD/APAD. Other types of plane alignment indicators may be used instead or in addition, including but not limited to straight lines, high water mark indicators, etc.

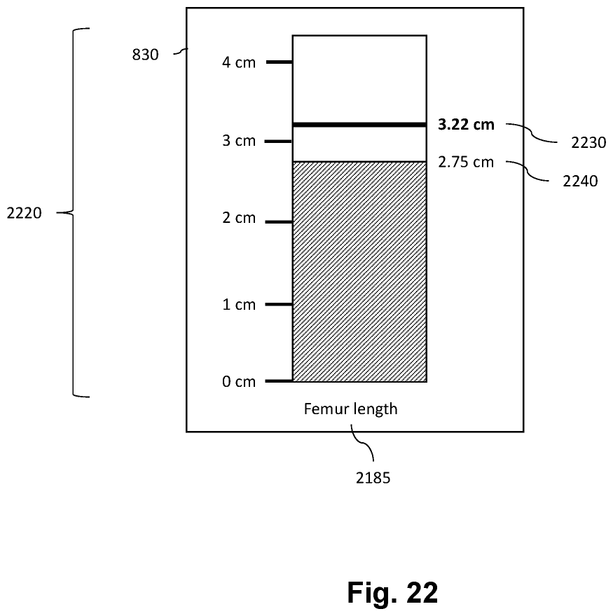

FIG. 22 is an example convergence progress display 830 of the automatic measurement point detection system, according to aspects of the present disclosure. In the example shown in FIG. 22, the convergence progress display 830 includes a measurement label 2185, a numerical scale 2220, a maximum measurement 2230, and a current measurement 2240. In an example, each time a new largest value for the current measurement is received, the maximum measurement is updated to the larger value. When a certain duration or a certain number of measurements have occurred without a larger value being received, the maximum measurement 2230 may be considered a converged and accurate measurement corresponding to the measurement label 2185.

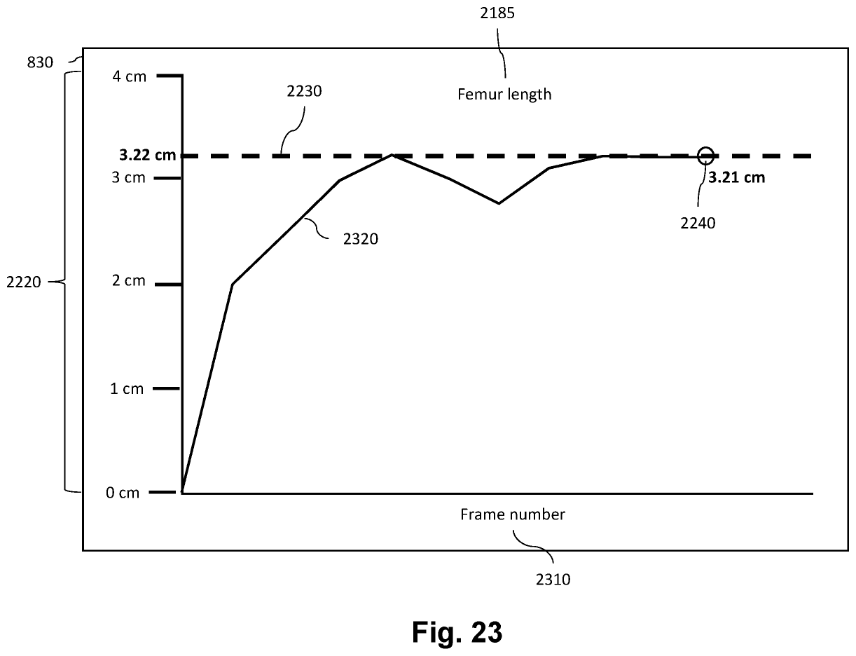

FIG. 23 is an example convergence progress display 830 of the automatic measurement point detection system, according to aspects of the present disclosure. In the example shown in FIG. 23, the convergence progress display 830 includes a measurement label 2185, a numerical scale 2220, a maximum measurement value 2230, and a current measurement value 2240. However, in this case, the progress of the current measurement over time is represented as a graph 2320, whose X-axis 2310 indicates a time or a frame number and whose Y-axis represents the numerical scale 2220. When a certain duration or a certain number of measurements have occurred without a current measurement value 2240 being received that is larger than the maximum measurement value 2230, the maximum measurement value 2230 may be considered a converged and accurate measurement corresponding to the measurement label 2185.

A number of variations are possible on the examples and aspects described above. For example, the systems, methods, and devices described herein are not limited to neonatal ultrasound applications. Rather, the same technology can be applied to images of other organs or anatomical systems such as the heart, brain, digestive system, vascular system, etc., or pathologies thereof. For example, for cardiac ultrasound, the left ventricle long axis and short axis distances may be measured, for vascular ultrasound, kidney width and length and/or liver length and width may be measured, and so forth. The measured anatomy could include any of the CSP, PLV, etc. (as shown in FIG. 10), or the stomach, spine, umbilical vein (as shown in FIG. 17), or any other anatomy of the patient for which the neural network has been trained.

Furthermore, the technology disclosed herein is also applicable to other medical imaging modalities obtained from a medical imaging device where 3D data is available, such as other ultrasound applications, camera-based videos, X-ray videos, and 3D volume images, such as computer aided tomography (CT) scans, magnetic resonance imaging (MRI) scans, optical coherence tomography (OCT) scans, or intravenous ultrasound (IVUS) pullback sequences. The technology described herein can be used in a variety of settings including emergency department, intensive care, inpatient, and out-of-hospital settings.

Accordingly, the logical operations making up the aspects of the technology described herein are referred to variously as operations, steps, objects, layers, elements, components, algorithms, or modules. Furthermore, it should be understood that these may occur or be performed or arranged in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the Automatic measurement point detection system. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the automatic measurement point detection system as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. A system, comprising:
a display; and
a processor configured for communication with the display and a medical imaging device, wherein the processor is configured to:
receive a first anatomical image frame obtained by the medical imaging device during live imaging;
identify, while the live imaging is ongoing, a plurality of first measurement points of an anatomical feature in the first anatomical image frame, wherein the identification of the first measurement points is performed by a first neural network trained to identify where a measurement point is located within an anatomical image frame such that the identification of the first measurement points is performed automatically without a user input to locate the plurality of first measurement points in the first anatomical image frame;
generate, based on the plurality of first measurement points, a first measurement value of the anatomical feature for the first anatomical image frame; and
output, to the display, a screen display based on the first measurement value.

2. The system of claim 1, wherein the processor is configured to:
identify, while the live imaging is ongoing, a plurality of second measurement points of the anatomical feature in a second anatomical image frame obtained by the medical imaging device during the live imaging; and
generate, based on the plurality of second measurement points, a second measurement value of the anatomical feature for the second anatomical image frame,
wherein the screen display is based on the first measurement and the second measurement.

3. The system of claim 2, wherein the second frame is obtained immediately after the first frame.

4. The system of claim 2,
wherein the processor is configured to determine, based on the first measurement value and the second measurement value, whether a convergence of measurement values has occurred,
wherein the processor is configured to provide the screen display based on the determination of whether the convergence of measurement values has occurred.

5. The system of claim 4, wherein the screen display comprises a progress of the convergence of measurement values.

6. The system of claim 4, wherein the processor is configured to determine whether the convergence of measurement values has occurred based on a difference between the first measurement and the second measurement.

7. The system of claim 5, wherein, to determine whether the convergence of measurement values has occurred, the processor is configured to determine whether the second measurement is smaller than the first measurement.

8. The system of claim 7,
wherein, if the convergence of measurement values has occurred, then the processor is configured to select, as a converged measurement value, the larger of the first measurement value or the second measurement value; and
wherein the screen display comprises the converged measurement value.

9. The system of claim 1, wherein the screen display comprises:
the first anatomical image frame; and
the plurality of first measurement points overlaid on the first anatomical image frame.

10. The system of claim 1, wherein the screen display comprises:
the first anatomical image frame; and
an indication of the anatomical feature overlaid on the first anatomical image frame.

11. The system of claim 1,
wherein the processor is configured to identify, in the first anatomical image frame, an anatomy comprising the anatomical feature,
wherein the processor is configured to generate the first measurement value based on the identification of the anatomy.

12. The system of claim 11, wherein the screen display comprises:
the first anatomical image frame; and
an indication of the anatomy overlaid on the first anatomical image frame.

13. The system of claim 11, wherein the identification of the anatomy is performed using a second neural network.

14. The system of claim 13, wherein the first neural network and the second neural network are the same neural network.

15. The system of claim 1, further comprising the medical imaging device.

16. A method, comprising:
receiving, with a processor in communication with a medical imaging device, an anatomical image frame obtained by the medical imaging device during live imaging;
identifying, while the live imaging is ongoing, a plurality of measurement points of an anatomical feature in the anatomical image frame, wherein the identification of the measurement points is performed by a neural network trained to identify where a measurement point is located within the anatomical image frame such that the identification of the first measurement points is performed automatically without a user input to locate the plurality of first measurement points in the anatomical image frame;
generating, based on the plurality of measurement points, a measurement value of the anatomical feature for the anatomical image frame;
outputting, to a display in communication with the processor, a screen display based on the measurement value.
